(19) United States
(12) Patent Application Publication
(10) Pub. No.: US 2022/0160455 A1
(43) Pub. Date: May 26, 2022

(54) IMAGING MARKER AND METHOD

(71) Applicant: Beekley Corporation, Bristol, CT (US)

(72) Inventor: Maeghan E. Traboulsi, Fairfax, VA (US)

(21) Appl. No.: 17/456,508

(22) Filed: Nov. 24, 2021

Related U.S. Application Data
(60) Provisional application No. 63/117,783, filed on Nov. 24, 2020.

(51) Int. Cl. A61B 90/00; A61N 5/10; G16H 20/40

(52) U.S. Cl. CPC A61B 90/39; A61N 5/1039; A61B 2090/3991; A61B 2090/3966; G16H 20/40

(57) ABSTRACT

An imaging marker is radiopaque at the level of radiation used during a radiation treatment planning and/or simulation. An adhesive layer is configured to releasably attach the imaging marker to a surface of a patient's skin. A radiolucent spacer is located between the adhesive layer and the radiopaque marker, and is radiolucent at the level of radiation used during the radiation treatment planning and/or simulation. The radiolucent spacer defines a thickness between the adhesive and substantially the entirety of the underside of the radiopaque marker of at least about 1 millimeter, and therefore spaces substantially the entirety of the underside of the radiopaque marker at least about 1 millimeter away from the skin.

### CROSS-REFERENCE TO PRIORITY AND RELATED APPLICATIONS

This application claims priority under 35 U.S.C. § 119 to U.S. Provisional Patent Application No. 63/117,783, filed Nov. 24, 2020, entitled “Imaging Marker and Method,” which is hereby expressly incorporated by reference in its entirety as part of the present disclosure. In addition, the co-pending application entitled “Imaging Marker and Method,” filed herewith by the Assignee of the present invention under Attorney Docket No. 97343.00238, is hereby expressly incorporated by reference in its entirety as part of the present disclosure.

### FIELD OF THE INVENTION

The present invention relates to imaging markers, such as for x-ray or computerized tomography (“CT”) imaging, and more particularly, to imaging markers used for radiation treatment planning and/or simulation.

### BACKGROUND INFORMATION

Radiation treating planning or simulation is a process used by a radiation therapist or therapy team to plan radiation treatments. The goal of the process is to precisely identify the area on a patient's body where the radiation treatment will be received. The simulation includes a CT scan of the area of the body to be treated with radiation. In certain cases, an MRI or a PET/CT scan also may be done. The images acquired during the scan are reconstructed and used to design a precise radiation treatment plan. The simulation is intended to ensure that subsequent radiation treatments will target the area(s) of concern, while minimizing effects on surrounding critical structures. Treatment planning usually involves positioning the patient's body, making marks and/or applying imaging markers to the skin, and taking imaging scans of the area of the body to be treated. Information from the imaging scans is used to precisely locate the treatment fields and create a “map” for the provider to design the treatment to fit the respective case. This helps the radiation oncologist determine the exact areas where the radiation treatment will be focused. To indicate the area where radiation should be aimed, the radiation therapist can use a combination of laser lights and marks on the body. This also ensures that the patient's body is precisely aligned and positioned properly for each treatment session. The type of mark(s) applied depends on the patient's cancer and treatment. In certain cases, marks are drawn on the patient's skin with a marker and/or imaging markers are releasably applied to the patient's skin with a pressure-sensitive adhesive. After simulation, details from the procedure are forwarded to medical radiation dosimetrists and medical physicists. These professionals perform highly technical calculations that are used to set the treatment machine (e.g., a linear accelerator). Typically, the dosimetrist and physicist work closely with the radiation oncologist to develop the treatment plan.

Radiation treatment machines, e.g., linear accelerators, resemble the simulator, but are larger. The patient is placed on the treatment table in the same position as in the simulator. Once properly positioned in place on the treatment table, a set of X-ray films or images are taken. These films or images are matched with the simulation images to ensure that the treatment is delivered the same way as it was simulated. Occasionally, the match is not optimal. In these cases, adjustments are made and checked by the attending physician. Once the films or images and positioning are confirmed, the radiation treatment is delivered. The radiation treatment planning or simulation process is critical to ensuring that the treatment be given as planned to avoid unnecessary toxicity to healthy tissue and to get the correct amount of radiation to the treatment area.

The assignee of the present invention manufactures and sells imaging markers that are used for radiation treatment planning or simulation. One such marker is a linear marker. Linear markers include a radiopaque portion that is linear shaped and is configured to be releasably attached to the patient's skin by an adhesive-backed substrate. The linear markers can be used to mark field borders, tangents, scars, and sarcomas. Preferably, the linear markers are flexible, contour to the skin, and clearly denote the area of concern in imaging and CT simulation without lifting or coming off the skin. One such linear marker is sold by Beekley Corp. under the brands CT-SPOT™ and S-SPOT™. Another type of imaging marker used in CT treatment planning is a crosshair-shaped marker. The marker includes a radiopaque portion that is crosshair shaped and is configured to be attached to a patient's skin by an adhesive-backed substrate. The distinct, crosshair marker shape can be used to clearly define the area of focus for a technician scrolling through multiple images in order to save time and improve accuracy. Such markers can be particularly useful in facilitating the ability of a radiation therapist to accurately locate the central axis or zero slice on a tumor field for treatment planning. Crosshair markers are used to mark 3-point set-ups, isocenters, and field borders, but also can be used to communicate in the images skin lesions, scars and specific points of pain on a patient to be included or excluded from the treatment field, or to mark the location of a port or drain on a patient. Beekley Corp. manufactures and sells such crosshair markers under the brand CT-SPOT™. Another type of imaging marker which is commonly used in radiation treatment planning or simulation is a pellet marker. Such markers include a radiopaque pellet or spherical marker mounted on an adhesive-backed substrate that is configured to be releasably attached to a patient's skin. The radiopaque pellet markers image “brightly” and therefore are resistant to radiation burnout in the resulting images. Such pellet markers can serve as guides for determining isocenters and 3-point set-ups, and can be used to identify critical structures and areas of concern. Beekley Corp. manufactures and sells pellet markers under the trademarks X-SPOT™, Y-SPOT™, Z-SPOT™, V-SPOT™, and CT-SPOT™.

One drawback associated with prior art imaging markers is that the underside of the radiopaque portion is located at or contiguous to the patient's skin, and therefore is located at or contiguous to the skin line in CT or other x-ray images. For example, in the above-described markers, the linear, crosshair or pellet-shaped radiopaque portion is mounted on a thin adhesive-backed substrate which, in turn, is releasably attached to the skin. As a result, the underside of the radiopaque portion is essentially located on the skin line in the CT or other x-ray images. During radiation treatment planning or simulation, the linear, crosshair or pellet-shaped radiopaque portion of the marker absorbs substantially more radiation than the surrounding tissue. However, because the underside of the radiopaque portion is located at the skin line, the treatment planning software may not be able to distinguish between the marker and the underlying skin and, as a result, may incorrectly determine that the patient's tissue is absorbing the radiation absorbed by the radiopaque marker. Thus, the prior art markers can cause the radiation treatment planning software to incorrectly calculate the radiation dose and in turn cause the software to create a “dose perturbation” or radiation “hot spot” at the location of the marker. Typical radiation treatment equipment includes software for automatically contouring the patient's body. In doing so, the software can pick-up the radiopaque marker as part of or included within the skin line of the patient's body. When this occurs, a dosimetrist or other professional is required to manually contour the patient's body at the location of the radiopaque marker. This may be achieved, for example, by manually cropping or carving out the radiopaque portion of the skin marker, such as by assigning the radiopaque portion of the marker the density of air. If the radiopaque portion were not manually carved or cropped out, it would give rise to a “dose perturbation” or radiation “hot spot.” If the latter were to occur, it would lead to an inaccurate or incorrect reading giving rise to a concern that the patient is receiving too much radiation at the location of the radiopaque marker.

Addressing the foregoing problem can increase the time and expense of the radiation treatment planning or simulation process.

It is an object of the present invention, and/or of embodiments thereof, to overcome one or more of the above-described drawbacks and/or disadvantages of the prior art.

### SUMMARY OF THE INVENTION

In accordance with one aspect of the present invention, an imaging marker configured for use in connection with an imager and radiation treatment planning and/or simulation software, comprises: (i) a marker that defines an underside and is substantially opaque or otherwise visible on an image of the marker taken by the imager in connection with a radiation treatment planning and/or simulation; (ii) an adhesive; and (iii) a spacer that is translucent or substantially invisible on the image of the marker taken by the imager in connection with the radiation treatment planning and/or simulation. The spacer is located between the adhesive and the marker. The spacer defines a thickness between the adhesive and substantially the entirety of the underside of the marker of at least about 1 millimeter. The adhesive is configured to releasably attach the imaging marker to a surface of a person's skin undergoing a radiation treatment planning and/or simulation at an interface of the imaging marker and the skin. The spacer spaces substantially the entirety of the underside of the marker a sufficient distance of at least about 1 millimeter away from the skin to substantially prevent the software from including the marker as part of the person or skin, or indicating that the person will absorb more radiation at the location of the marker than the person otherwise would absorb at that location during the radiation treatment.

In some embodiments of the present invention, the imager generates images by transmitting radiation. In some such embodiments, the marker is formed by at least one radiopaque portion that is substantially radiopaque at a level of radiation used by the imager in connection with the radiation treatment planning and/or simulation, and the spacer is substantially radiolucent at the level of radiation used by the imager in connection with the radiation treatment planning and/or simulation. In some embodiments of the present invention, the marker is linear shaped, cross-shaped or pellet shaped. In some such imaging markers, the marker consists of a single pellet.

In some embodiments of the present invention, the spacer defines a thickness between the adhesive and the underside of the marker of at least about 1-⅕ millimeters. In some such embodiments, the spacer defines a thickness between the adhesive and the underside of the marker of at least about 1-⅖ millimeters. In some such embodiments, the spacer defines a thickness between the adhesive and the underside of the marker of at least about 1-⅗ millimeters. Preferably, in each such embodiment, the thickness between the adhesive and the underside of the marker is substantially uniform.

In some embodiments of the present invention, the marker is linear shaped and defines an elongated axis. The spacer defines an axially-elongated portion that extends along the elongated axis between the linear marker and the adhesive. The spacer further defines a plurality of laterally-extending portions located on opposite sides of the elongated axis relative to each other. At least a portion of a plurality of the laterally-extending portions are axially spaced relative to each other along the elongated axis. In some such embodiments, the linear marker is flexible, and the spacer is configured to flex at least between the axially-spaced, laterally-extending portions to thereby allow the spacer to flex with the marker. In some such embodiments, the spacer defines a plurality of pairs of laterally-extending portions that extend laterally on opposite sides of the elongated axis relative to each other, and relatively narrow-width portions located between axially-spaced pairs of laterally-extending portions.

In some embodiments of the present invention, the marker is cross-shaped, is defined by two intersecting linear-shaped portions, and each linear-shaped portion defines an elongated axis. The spacer extends along each elongated axis and is located between the respective linear-shaped portion and adhesive layer. In other embodiments of the present invention, the marker consists essentially of a single pellet, and the spacer is located between the pellet and the adhesive.

In some embodiments of the present invention, the imaging marker is mounted on a releasable liner, and the releasable liner is releasably attached to the adhesive. In some such embodiments, the marker defines an elongated axis and a continuous linear shape extending along the elongated axis, and the releasable liner defines an axially-elongated shape extending along the elongated axis of the linear marker. In some such embodiments, the linear marker and releasable backing are configured to be torn, cut or separated, such as by scissors or another cutting instrument or tool, at desired locations to form individual imaging markers therefrom at desired lengths. In some embodiments of the present invention, a plurality of imaging markers are mounted and spaced relative to each other on the releasable liner, and the adhesive of each imaging marker is releasably attached to the releasable liner.

In some embodiments of the present invention, the spacer is formed of a radiolucent foam. In some such embodiments, the foam is a thermoplastic or thermoset foam. In some embodiments of the present invention, the marker is made of a thermoplastic including a filler of sufficient density to make the marker substantially radiopaque at a level of radiation used in connection with a radiation treatment planning and/or simulation.

In accordance with another aspect of the present invention, an imaging marker configured for use in connection with an imager and radiation treatment planning and/or simulation software, comprises: (i) first means that is substantially opaque or visible on an image of the marker taken by the imager in connection with a radiation treatment planning and/or simulation for forming an image thereof in connection with the radiation treatment planning and/or simulation; (ii) second means for releasably attaching the imaging marker to a surface of the skin of a person undergoing radiation treatment planning and/or simulation; and (iii) third means located between the first and second means that is translucent or substantially invisible on the image of the marker taken by the imager in connection with the radiation treatment planning and/or simulation for spacing substantially the entirety of the underside of the first means a sufficient distance of at least about 1 millimeter away from the skin for substantially preventing the software from including the first means as part of the person or skin, or indicating that the person will absorb more radiation at the location of the first means than the person otherwise would absorb at that location during the radiation treatment

In some embodiments of the present invention, the first means is linear shaped, cross-shaped or consists essentially of a single substantially spherical shape. In some such embodiments, the first means is a substantially radiopaque marker, the second means is an adhesive, and the third means is a substantially radiolucent spacer. In some such embodiments, the marker is made of a thermoplastic including a filler of sufficient density to make the marker substantially radiopaque at the level of radiation used in connection with the radiation treatment planning and/or simulation.

In accordance with another aspect, the present invention is directed to a method comprising: (i) releasably attaching to a surface of the skin of a person an adhesive portion of an imaging marker, wherein the imaging marker includes a marker portion that is substantially opaque or visible on an image of the marker taken by an imager in connection with a radiation treatment planning and/or simulation, and a spacer located between the adhesive and the marker portion that is translucent or substantially invisible on the image of the marker taken by the imager in connection with the radiation treatment planning and/or simulation; (ii) imaging with the imager the marker portion of the imaging marker and at least a portion of the person in connection with the radiation treatment planning and/or simulation such that the marker portion is substantially opaque or visible and the spacer is translucent or substantially invisible on the image of the marker taken by the imager; and (iii) during the imaging of step (ii), spacing with the spacer substantially the entirety of the underside of the marker portion a sufficient distance of at least about 1 millimeter away from the skin and substantially preventing the software from including the marker as part of the person or skin, or indicating that the person will absorb more radiation at the location of the marker portion than the person otherwise would absorb at that location during radiation treatment.

In some embodiments of the present invention, the imaging includes transmitting radiation through the imaging marker at a level at which the marker portion is substantially radiopaque to the transmitted radiation and the spacer is substantially radiolucent to the transmitted radiation. In some embodiments of the present invention, the marker portion is linear shaped, and the method further comprises marking with the imaging marker a field border, tangent, scar, match line, outer canthus, node, sarcoma and/or a treatment area. In other embodiments of the present invention, the marker portion is cross shaped, and the method further comprises marking with the imaging marker a central axis or zero slice on a tumor field. In other embodiments of the present invention, the marker portion is a single pellet, and the method further comprises marking with the imaging marker an isocenter, a point in a multiple point set up, an underlying structure, or an area of concern.

One advantage of the present invention, and/or of one or more embodiments thereof, is that the spacer or third means, such as a radiolucent spacer, defines a thickness of at least about 1 millimeter between the adhesive or second means and substantially the entirety of the underside of the marker or first means, such as a radiopaque marker, and therefore spaces substantially the entirety of the underside of the marker or first means at least about 1 millimeter away from the skin. As a result, the automatic body contouring or other software of the radiation therapy equipment is substantially prevented from picking-up the marker or first means as part of or included within the skin line of the patient's body and giving rise to a “dose perturbation” or radiation “hot spot” at the location of the marker or first means. Accordingly, the need to manually crop or carve out a radiopaque marker from the skin line encountered with the above-described prior art markers, and the associated time, expense and inconvenience of doing so, are substantially avoided.

Other advantages of the present invention, and/or of the embodiments thereof, will become more readily apparent in view of the followed detailed description of embodiments of the invention and accompanying drawings.

### BRIEF DESCRIPTION OF THE DRAWINGS

FIG. 1 is a partial, perspective view of a linear-shaped marker embodying the present invention;

FIG. 2 is a top plan view of the linear marker of FIG. 1;

FIG. 3 is a cross-sectional view of the linear marker of FIG. 1 taken along line A-A of FIG. 2;

FIG. 4 is a partial, perspective view of the linear marker of FIG. 1 removed from its releasable backing and applied to a patient's skin;

FIG. 5 is an illustration of an exemplary CT or other x-ray image of the marker of FIG. 4 applied to the patient's skin and illustrating the manner in which the radiolucent spacer spaces the radiopaque portion of the marker at least about 1 millimeter away from the patient's skin line;

FIG. 6 is a perspective view of a crosshair-shaped marker embodying the present invention; and

FIG. 7 is a perspective view of a pellet-shaped marker embodying the present invention.

### DETAILED DESCRIPTION OF EMBODIMENTS OF THE INVENTION

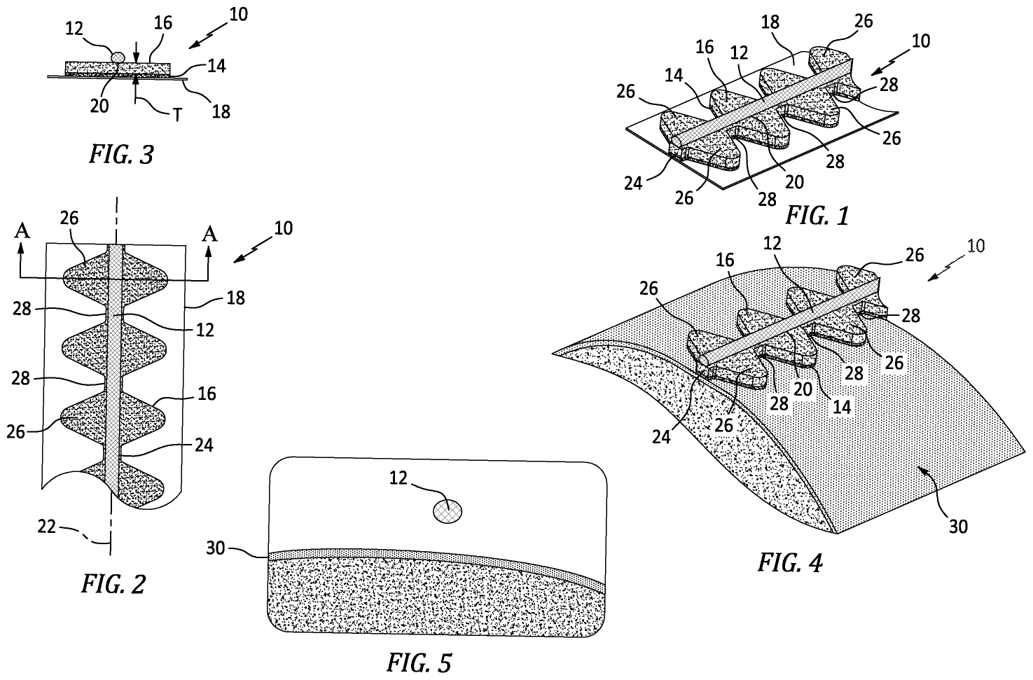

In FIG. 1, an imaging marker embodying the present invention is indicated generally by the reference numeral 10. The imaging marker 10 comprises a radiopaque marker or portion 12, an adhesive layer or coating 14, and a radiolucent or radiotransparent spacer 16 located between the adhesive and the radiopaque marker. As shown in FIGS. 1-3, the adhesive 14 releasably attaches the imaging marker 10 to a releasable liner 18. The radiopaque marker 12 is radiopaque at the level of radiation used during the radiation treatment planning and/or simulation. The radiolucent spacer 16, on the other hand, is radiolucent at the level of radiation used during the radiation treatment planning and/or simulation. Accordingly, the radiopaque marker 12 is opaque or otherwise visible in an x-ray, CT or other type of image of the marker, whereas the radiolucent spacer 16 is translucent or invisible on the image. As shown typically in FIG. 4, the adhesive layer 14 is configured to releasably attach the imaging marker 10 to a surface of a patient's skin undergoing the radiation treatment planning and/or simulation at an interface of the imaging marker and the skin. In the illustrated embodiment, the adhesive 14 is in the form of a coating that underlies the radiolucent spacer 16. The adhesive coating 14 is preferably pressure sensitive in that it releasably attaches the imaging marker to a person's skin when slight pressure is applied thereto. The adhesive may take the form of any of numerous different types of adhesives or other substances that are currently known, or that later become known for releasably attaching an imaging marker to a person's skin.

The radiopaque marker 12 defines an underside 20, and the radiolucent spacer 16 defines a thickness “T” between the adhesive 14 and substantially the entirety of the underside 20 of the radiopaque marker. In the illustrated embodiment, the thickness T is at least about 1 millimeter. Accordingly, in the illustrated embodiment, the radiolucent spacer 16 spaces substantially the entirety of the underside 20 of the radiopaque marker 12 at least about 1 millimeter away from the skin. In some embodiments of the present invention, the thickness T is at least about 1-⅕ millimeters, preferably is at least about 1-⅖ millimeters, and is more preferably at least about 1-⅗ millimeters. The maximum thickness T of the radiolucent spacer 16 is generally not as critical as its minimum thickness. In other words, the radiolucent spacer should define a sufficient thickness T in order to space the underside of the radiopaque marker away from the skin line and thereby prevent the software used in connection with the radiation treatment planning and/or simulation from mistakenly including the radiopaque marker as part of the patient's skin, or otherwise mistakenly indicating that the patient is absorbing radiation absorbed by the radiopaque marker. Accordingly, the radiolucent spacer should define a thickness T that is sufficient to perform this function; however, the thickness T may be greater than the minimum thickness required to perform this function. On the other hand, if the thickness T of the radiolucent spacer is too great, it could interfere with the ability to handle the marker. For example, if the thickness T of a line-shaped radiolucent spacer is too great, it may reduce the ease of handling the imaging marker and/or interfere with the ability to bend or otherwise deform the imaging marker into a required or desired shape. In addition, if the thickness T of a radiolucent spacer is too great, it may prevent packaging a required or desired number of imaging markers into a conventional or commonly-sized package. Accordingly, in some embodiments, the radiolucent spacer defines a thickness T that is sufficient to perform the foregoing function, but nevertheless defines a relatively low profile to facilitate handling, packaging, or otherwise prevent the marker from creating an undesirable obstruction or profile during use. In some such embodiments, the thickness T of the radiolucent spacer is within the range of (i) about 1 to about 1.6 millimeters, (ii) about 1 to about 2.4 millimeters, (iii) about 1 to about 3.2 millimeters, or (iv) about 1 to about 4 millimeters. As shown in FIGS. 1-4, in the illustrated embodiments, the thickness T of the radiolucent spacer is substantially uniform throughout.

As shown typically in FIG. 2, the radiopaque marker 12 is linear shaped and defines an elongated axis 22. As shown in FIGS. 1 and 4, the radiolucent spacer 16 defines an axially-elongated portion 24 that extends along the elongated axis 22 between the linear radiopaque marker 12 and adhesive-backed substrate 14. The radiolucent spacer 16 defines a plurality of pairs of laterally-extending portions 26, 26 that extend laterally on opposite sides of the elongated axis 22 relative to each other, and relatively narrow-width portions 28, 28 located between axially-spaced pairs of laterally-extending portions. As can be seen, the laterally-extending portions 26, 26 are axially spaced relative to each other along the elongated axis by the relatively narrow-width portions 28, 28. In the illustrated embodiment, both the radiopaque marker 12 and radiolucent spacer 16 are flexible in order to allow the marker to conform its shape to the contour of the patient's skin to which it is releasably attached and allow it to otherwise take any desired shape. The relatively narrow-width portions 28, 28 located between the laterally-extending pairs 26, 26 allow the laterally-extending pairs to flex relative to each other about the elongated axis 22 in order to flex or deform the shape of the marker as needed. For example, the linear shape of the imaging marker 10 may be flexed and/or deformed as needed in order to mark field borders, tangents, scars, or sarcomas and/or to otherwise clearly denote an area of concern in imaging or CT simulation without lifting or coming off the skin. The radiolucent spacer 16 is sufficiently flexible and/or moldable to form desired shapes as dictated by the anatomy or anatomical features of each patient, and as indicated above, defines a thickness T that is preferably substantially uniform throughout. In the illustrated embodiment, the radiolucent spacer 16 is formed of a thermoplastic or thermoset foam, such as a closed cell, polyethylene foam. The radiopaque marker 12 is of sufficient density so that it is radiopaque at the level of radiation used during the radiation treatment planning and/or simulation and is sufficiently flexible and/or moldable to form desired shapes as dictated by the anatomy or anatomical features of each patient. In the illustrated embodiment, the radiopaque portion 12 is made of an extruded thermoplastic including a filler of sufficient density to make the radiopaque portion radiopaque at the level of radiation used during the radiation treatment planning and/or simulation.

As may be recognized by those of ordinary skill in the pertinent art based on the teachings herein, the radiopaque marker 12 and the radiolucent spacer 16 may be made of any of numerous different materials that are currently known, or that later become known, for purposes of performing the functions of these elements or components of the imaging marker. For example, the radiolucent spacer may be formed of an open cell or closed cell foam made of any of numerous different materials that are currently known or that later become known. In addition, the radiolucent spacer need not be a foam, but rather can be made of another radiolucent material. Similarly, the radiopaque portion 12 may be non-metallic, as described above, or may be metallic. For example, the metallic radiopaque portion could be formed by a flexible, deformable metal wire, such as a steel wire. If non-metallic, the body of the radiopaque marker 12 and its radiopaque filler may be made any of numerous different materials that are currently known or that later become known. In addition, the imaging marker is configurable to work with any type of imager, imaging process or imaging system. When configured for each such imager, imaging process or system, the marker is configured to be opaque, partially radiopaque, partially radiolucent or radiotransparent, or otherwise visible on an image of the marker taken by such imager, process or system, whereas the spacer is configured to be translucent or invisible on the image. For example, if configured for use with magnetic resonance (“MR”) imaging (also referred to as MRI or NMR imaging), the marker 12 is made of or includes a material that is opaque or visible on an MR image, whereas the spacer 16 is made of or includes a material that is not visible or is translucent on the MR image. Accordingly, the imaging markers can be used with any of numerous different imagers, imaging processes or imaging systems, that are currently known, or that later become known, and may be made of any of numerous different materials required by such imagers, processes or systems in order to cause the marker or marker portion to be opaque or visible in images of the marker, and the spacer to be translucent or invisible in such images.

In the illustrated embodiment, the laterally-extending pairs 26, 26 take the form of tabs that are approximately triangular-shaped. However, as may be recognized by those of ordinary skill in the pertinent art based on the teachings herein, the laterally-extending portions may take any of numerous other shapes, such as semi-circular or semi-oval shapes, and the laterally-extending tabs or other portions need not be laterally aligned, as shown, but may be axially offset relative to each other.

The radiolucent spacer 16 and its underlying adhesive 14 allow the marker to conform to the contour of the patient's skin and otherwise flex or deform into a desired shape in order to releasably attach the marker to the patient's skin in the desired shape without coming off of the skin during radiation treatment planning and/or simulation. As shown in FIGS. 1-3, the marker 10 is supplied on a releasable backing 18 in strip form that allows the marker to be removed therefrom and, in turn, adhesively attached to a patient's skin. In use, the marker 10 is cut to the desired length with, for example, a pair of scissors (not shown). A pair of scissors or other desired cutting instrument may be used to easily cut through the releasable backing 18 and the marker 10 in order to cut the desired length of marker for a respective application. The marker 10 can be supplied on its releasable backing 18 in a roll form (not shown) and dispensed and cut, torn or otherwise separated to desired lengths as needed while preserving the remainder of the roll for future use.

As shown typically in FIG. 5, the marker 10 is cut to the desired length, is removed from its piece of releasable backing 18, is deformed or otherwise molded into a desired shape, as needed, and is releasably attached to the patient's skin with the adhesive 14. The flexible nature of the radiopaque marker 10 and adhesive-backed radiolucent spacer 16 allow the marker to conform to the contour of the patient's skin and otherwise conform to or mark the anatomical features of interest. As shown typically in FIG. 5, the radiopaque marker 12 is substantially radiopaque at the level of radiation used during the radiation treatment planning and/or simulation, whereas the radiolucent spacer 16 and adhesive backing 14 are substantially radiolucent at the level of radiation used during the radiation treatment planning and/or simulation. As a result, and as shown typically in FIG. 5, only the radiopaque marker 12 is visible on a CT or other image taken during radiation treatment planning and/or simulation. In addition, as also shown typically in FIG. 5, the radiolucent spacer 16 spaces substantially the entirety of the underside of the radiopaque marker 12 at least about 1 millimeter away from an interface of the imaging marker and the surface of the skin 30 of the patient. As a result, the imaging marker 10 avoids the need to manually crop or carve out the radiopaque marker from the skin line, and the associated time, expense and inconvenience of doing so, as encountered in the above-described prior art.

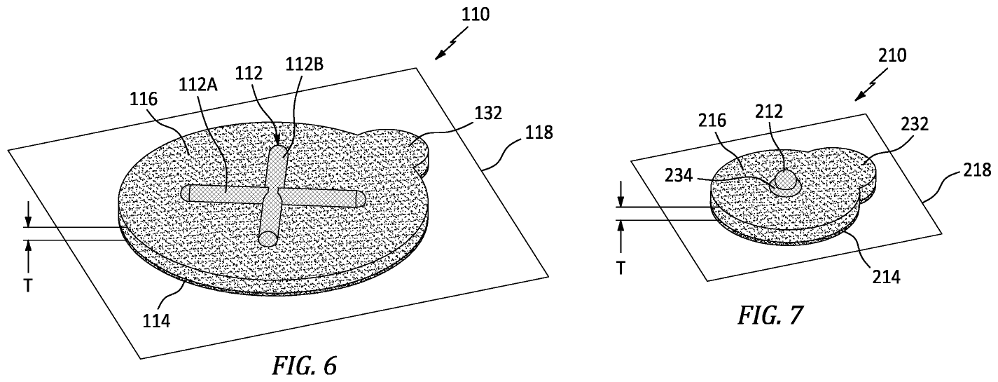

In FIG. 6, another embodiment of an imaging marker is indicated generally by the reference numeral 110. The imaging marker 110 is similar to the imaging marker 10 described above, and therefore like reference numerals preceded by the numeral “1” are used to indicate like elements. Accordingly, the descriptions of the like elements above are hereby expressly incorporated by reference as part of the descriptions of the elements of this embodiment. A primary difference of the imaging marker 110 as compared to the imaging marker 10 is that it includes a crosshair-shaped radiopaque marker or portion 112 rather than the linear-shape of the radiopaque marker 10 described above. The radiopaque crosshair 112 includes two mutually perpendicular, linear radiopaque portions 112A, 112B. The radiopaque crosshair 112 is mounted on a radiolucent spacer 116 which is backed with a pressure-sensitive adhesive 114. The adhesive-backed substrate 114 is releasably attached to a releasable backing 118. As shown in FIG. 6, the radiolucent spacer 116 and underlying adhesive 114 are substantially circular shaped and define a tab 132 extending radially outwardly therefrom. The tab 132 facilitates the ability of a user to grip the imaging marker 110 and remove it from the releasable backing 118. If desired, the releasable backing 118 may be die cut beneath the tab 132 to facilitate the ability of a user to manually grip the tab, lift the tab away from the releasable backing 118, and in turn apply the marker to a patient's skin. Otherwise, the radiopaque marker 112, radiolucent spacer 116, and adhesive 114 may define the same thicknesses and be made of the same materials as the radiopaque marker 12, radiolucent spacer 16 and adhesive 14, respectively, described above. The releasable liner 118 may be provided in strip form (i.e., in an axially-elongated strip), and a plurality of imaging markers 110 can be mounted on the strip axially spaced relative to each other. The strip 118 with plural markers 110 may be provided in roll form such that the markers 110 may be dispensed and removed from the releasable liner as needed and the remaining markers may be retained on the roll for future use.

The distinct, crosshair shape of the marker 112 can be used to clearly define an area of focus for a technician scrolling through multiple images in order to save time and improve accuracy. The crosshair marker 112 also can be used, for example, to accurately locate the central axis or zero slice on a tumor field for treatment planning, to mark 3-point set-ups, isocenters, and field borders, to communicate in the images skin lesions, scars and specific points of pain on a patient to be included or excluded from the treatment field, or to mark the location of a port or drain on a patient. Although the adhesive-backed radiolucent spacer 116 is shown as substantially circular shaped, and the tab 132 is substantially semi-circular shaped, they can take any of numerous different shapes and/or configurations that are currently known, or that later become known.

In FIG. 7, another embodiment of an imaging marker is indicated generally by the reference numeral 210. The imaging marker 210 is similar to the imaging markers 10 and 110 described above, and therefore like reference numerals preceded by the numeral “2”, or preceded by the numeral “2” instead of the numeral “1”, are used to indicate like elements. Accordingly, the descriptions of the like elements above are hereby expressly incorporated by reference as part of the descriptions of the elements of this embodiment. A primary difference of the imaging marker 210 as compared to the imaging markers 10 and 100 described above, is that the imaging marker 210 includes a single, substantially spherical-shaped radiopaque marker or portion 212 rather than the linear- or crosshair-shaped radiopaque portions described above. In the illustrated embodiment, the single, substantially spherical-shaped marker 212 is in the form of a non-metallic pellet. Accordingly, the marker 212 may be made of the same or similar materials as the markers 12 or 112 described above. However, as may be recognized by those of ordinary skill in the pertinent art based on the teachings herein, the marker 212 may be formed of any of numerous other materials and/or take any of numerous other shapes that are currently known, or that later become known, such as an oval shape. The spherical-shaped marker 212 is fixedly secured by an adhesive 234 to the approximate center of a radiolucent spacer 216 which includes an underlying adhesive coating 214. The adhesive 214 is releasably attached to a releasable backing 218. As shown in FIG. 7, the adhesive-backed radiolucent spacer 216 is substantially circular shaped and defines a tab 232 extending laterally outwardly therefrom. The tab 232 facilitates the ability of a user to grip the imaging marker 210 and remove it away from the releasable backing 218. If desired, the releasable backing 218 may be die cut beneath the tab 232 to facilitate the ability of a user to manually grip the tab, lift the tab away from the releasable backing 218, and apply the marker to a patient's skin. Otherwise, the adhesive-backed radiolucent spacer 216 may define the same thicknesses and be made of the same materials as the adhesive-backed radiolucent spacer 16 or 116, as described above. The releasable liner 218 may be provided in strip form (i.e., in an axially-elongated strip), and a plurality of imaging markers 210 can be mounted on the strip axially spaced relative to each other. The strip 218 with plural markers 210 thereon may be provided in roll form such that the markers 210 may be dispensed and removed from the releasable liner as needed and the remaining markers may be retained on the roll for future use.

As may be recognized by those of ordinary skill in the pertinent art based on the teachings herein, numerous modifications, changes and/or additions may be made to the above-described and other embodiments of the present invention without departing from the scope of the invention as defined in the claims. For example, the imaging markers may include additional components or layers or fewer components or layers, may be made of any of numerous different materials, and/or may take any of numerous different shapes and/or configurations, that are currently known or that later become known. In addition, the shapes and/or configurations of the marker portion and/or spacer, and/or their materials of construction, may be changed as required, such as to adjust their imaging characteristics (e.g. their degree of radiopacity or radiolucency or radiotransparency), to work with different types of imagers, imaging processes or imaging systems, that are currently known or that later become known, and including without limitation, x-ray, CT, PET, MRI or NMR imaging processes and systems. Accordingly, the configurations and/or materials of the components may be selected as dictated by the particular imager, imaging process or imaging system with which the imaging marker is to be used, so that each component is opaque, partially radiopaque, partially radiolucent or radiotransparent, or otherwise visible on the image, or is translucent or invisible on the image, as disclosed herein. Accordingly, this detailed description of embodiments is to be taken in an illustrative, as opposed to a limiting sense.

What is claimed is:

1. An imaging marker configured for use in connection with an imager and radiation treatment planning and/or simulation software, comprising:
a marker that is substantially opaque or visible on an image of the marker taken by the imager in connection with a radiation treatment planning and/or simulation, wherein the marker defines an underside;
an adhesive; and
a spacer that is translucent or substantially invisible on the image of the marker taken by the imager in connection with the radiation treatment planning and/or simulation, wherein the spacer is located between the adhesive and the marker, the spacer defines a thickness between the adhesive and substantially the entirety of the underside of the marker of at least about 1 millimeter, the adhesive is configured to releasably attach the imaging marker to a surface of a person's skin undergoing the radiation treatment planning and/or simulation at an interface of the imaging marker and the skin, and the spacer spaces substantially the entirety of the underside of the marker a sufficient distance of at least about 1 millimeter away from the skin to substantially prevent the software from including the marker as part of the person or skin, or indicating that the person will absorb more radiation at the location of the marker than the person otherwise would absorb at that location during the radiation treatment.

2. An imaging marker as defined in claim 1, wherein the imager generates images by transmitting radiation, the marker is formed by at least one radiopaque portion that is substantially radiopaque at a level of radiation used by the imager in connection with the radiation treatment planning and/or simulation, and the spacer is substantially radiolucent at the level of radiation used by the imager in connection with the radiation treatment planning and/or simulation.

3. An imaging marker as defined in claim 1, wherein the marker is linear shaped, cross-shaped or pellet shaped.

4. An imaging marker as defined in claim 3, wherein the marker is pellet shaped and consists of a single pellet.

5. An imaging marker as defined in claim 1, wherein the spacer extends between the adhesive and the marker and defines a thickness between the adhesive and the underside of the marker of at least about 1-⅕ millimeters, at least about 1-⅖ millimeters, or at least about 1-⅗ millimeters.

6. An imaging marker as defined in claim 1, wherein the thickness between the adhesive and the underside of the marker is substantially uniform.

7. An imaging marker as defined in claim 1, wherein the marker is linear shaped and defines an elongated axis, and the spacer defines an axially-elongated portion extending along the elongated axis between the linear marker and the adhesive, and a plurality of laterally-extending portions, wherein a plurality of the laterally-extending portions are located on opposite sides of the elongated axis relative to each other, and at least a portion of a plurality of the laterally-extending portions are axially spaced relative to each other along the elongated axis.

8. An imaging marker as defined in claim 7, wherein the linear marker is flexible, and the spacer is configured to flex at least between the axially-spaced, laterally-extending portions to thereby allow the spacer to flex with the marker.

9. An imaging marker as defined in claim 8, wherein the spacer defines a plurality of pairs of laterally-extending portions extending laterally on opposite sides of the elongated axis relative to each other, and relatively narrow-width portions located between axially-spaced pairs of laterally-extending portions.

10. An imaging marker as defined in claim 1, wherein the marker is cross-shaped, is defined by two intersecting linear-shaped portions, each linear-shaped portion defines an elongated axis, and the spacer extends along each elongated axis and is located between the respective linear-shaped portion and the adhesive.

11. An imaging marker as defined in claim 1, wherein the adhesive defines an adhesive coating underlying the spacer.

12. An imaging marker as defined in claim 1, wherein the marker consists essentially of a single pellet and the spacer is located between the pellet and the adhesive.

13. An imaging marker as defined in claim 1, wherein the imaging marker is mounted on a releasable liner, and the releasable liner is releasably attached to the adhesive.

14. An imaging marker as defined in claim 13, wherein the marker defines an elongated axis and a continuous linear shape extending along the elongated axis, and the releasable liner defines an axially-elongated shape extending along the elongated axis of the linear marker.

15. An imaging marker as defined in claim 14, wherein the linear marker and releasable backing are configured to be torn, cut or separated at desired locations to form individual imaging markers therefrom at desired lengths.

16. An imaging marker as defined in claim 2, wherein the spacer is formed of a foam.

17. An imaging marker as defined in claim 16, wherein the foam is a thermoplastic or thermoset foam.

18. An imaging marker configured for use in connection with an imager and radiation treatment planning and/or simulation software, comprising:
first means that is substantially opaque or visible on an image of the marker taken by the imager in connection with a radiation treatment planning and/or simulation for forming an image thereof during the radiation treatment planning and/or simulation;
second means for releasably attaching the imaging marker to a surface of the skin of a person undergoing the radiation treatment planning and/or simulation; and
third means located between the first and second means that is translucent or substantially invisible on the image of the marker taken by the imager in connection with the radiation treatment planning and/or simulation for spacing substantially the entirety of the underside of the first means a sufficient distance of at least about 1 millimeter away from the skin for substantially preventing the software from including the first means as part of the person or skin, or indicating that the person will absorb more radiation at the location of the first means than the person otherwise would absorb at that location during the radiation treatment.

19. An imaging marker as defined in claim 18, wherein the first means is a substantially radiopaque marker, the second means is an adhesive, and the third means is a substantially radiolucent spacer.

20. An imaging marker as defined in claim 19, wherein the marker is linear shaped or cross shaped, or consists of a single, substantially spherical-shaped pellet.

21. A method comprising:
releasably attaching to a surface of the skin of a person an adhesive portion of an imaging marker, wherein the imaging marker includes a marker portion that is substantially opaque or visible on an image of the marker taken by an imager in connection with a radiation treatment planning and/or simulation, and a spacer located between the adhesive and the marker portion that is translucent or substantially invisible on the image of the marker taken by the imager in connection with the radiation treatment planning and/or simulation;
imaging with the imager the marker portion of the imaging marker and the person in connection with the radiation treatment planning and/or simulation such that the marker portion is substantially opaque or visible and the spacer is translucent or substantially invisible on the image of the marker taken by the imager; and
during the imaging of step (ii), spacing with the spacer substantially the entirety of the underside of the marker portion a sufficient distance of at least about 1 millimeter away from the skin and substantially preventing the software from including the marker as part of the person or skin, or indicating that the person will absorb more radiation at the location of the marker portion than the person otherwise would absorb at that location during radiation treatment.

22. A method as defined in claim 21, wherein the imaging includes transmitting radiation through the imaging marker at a level at which the marker portion is substantially radiopaque to the transmitted radiation and the spacer is substantially radiolucent to the transmitted radiation.

23. A method as defined in claim 21, wherein the marker portion is linear shaped, and further comprising marking with the imaging marker a field border, tangent, scar, match line, outer canthus, node, sarcoma and/or a treatment area.

24. A method as defined in claim 21, where the marker portion is cross shaped, and further comprising marking with the imaging marker a central axis or zero slice on a tumor field.

25. A method as defined in claim 21, wherein the marker portion is a single pellet, and further comprising marking with the imaging marker an isocenter, a point in a multiple point set up, an underlying structure, or an area of concern.

26. A method as defined in claim 21, further comprising substantially preventing dose perturbation at the interface of the imaging marker and the surface of the skin.
